(19) United States
(12) Patent Application Publication
(10) Pub. No.: US 2010/0263670 A1
(43) Pub. Date: Oct. 21, 2010

(54) Resuscitator

(76) Inventor: Richard Pearce, Tiburon, CA (US)

Correspondence Address: Richard Pearce, 2288 Spanish Trail RD, Tiburon, CA, 94920, US

(21) Appl. No.: 12/386,255

(22) Filed: Apr. 16, 2009

(51) Int. Cl. A61M 16/08

(52) U.S. Cl. 128/205.14

(57) ABSTRACT

A resuscitator with a rigid top plate, a rigid bottom plate and a rigid bottom plenum assembly. The top plate is hingeldy joined at its forward end to the bottom plate. A flexible accordion member is located between the top plate and the bottom assembly forming a standard air pump. When a person pushes down on the top plate, air is forced out of and outflow tube located on the top plate. An internal spring returns the top plate to its original position and in doing so causes air to be drawn into the plenum assembly. A top plate travel limiting member, such as a cord, includes a plurality of spaced protuberances that act as restraining members that can be retained under a slot in the side of the plenum assembly thereby adjusting the travel distance of the accordion member and controlling the resulting volume of air output to a patient being resuscitated.

### BACKGROUND OF THE INVENTION

(1) Field of the Invention

This invention relates generally to the field of emergency equipment and more specifically to a resuscitator.

(2) Description of the Related Art

Resuscitators are a known class of devices that are used by professionals such as medics, fire fighters, police and hospital personell to help revive a person who has stopped breathing. Essentially, they take the place of mouth to mouth resuscitation.

One typical resuscitator is made by Ambu A/S of Ballerup, Denmark. The Ambu Spur II Resuscitator is comprised of a football shaped silicon rubber bladder having an air input aperture on one side and an air exit aperture including a tube and face mask on the opposite side. Internal check valves create an air pumping action each time the rubber bladder is squeezed thereby introducing a volume of air to the face mask and into the patient's airway.

Current resuscitator designs work relatively well and have saved countless lives, however, there is a deficiency in the prior technology. When a user squeezes the silicone rubber bladder, there is no guarantee regarding the consistency of the volume of air that is being delivered to the patient with each squeezing cycle. It all depends on how hard the user squeezes the bladder. Furthermore, current resuscitator manufacturers have found it necessary to make three different sizes of bladder assemblies. One for infants, one for children and one for adults. This means that an emergency vehicle has to carry three different items with them at any time, and finding the correct size for the situation can add additional stress to what is usually an already chaotic and stressful situation.

### BRIEF SUMMARY OF THE INVENTION

The primary object of the invention is to provide a resuscitator that allows the user to deliver a precise volume of air to ventilate a patient no matter what hand size the rescuer may have.

Another object of the invention is to provide a resuscitator that whose volume per stroke can be adjusted to accommodate an infant, a child or an adult.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a resuscitator comprising: a rigid top plate, a rigid bottom plate, a rigid bottom sub plate separated from and parallel to said bottom plate, a continuous rigid side surround panel that joins the perimeter edges of said bottom plate and said bottom sub plate forming an air flow plenum assembly, said top plate hingely joined at its forward end to said bottom plate, a flexible accordion member, an air intake check valve, an air outflow check valve, an air outflow tube fixedly attached to the top surface of said top plate, a compression spring, a top plate travel limiting member, the underside of said top plate joined to the upper side of said bottom plate by said accordion member, said top plate including an aperture covered on its top surface by said air outflow check valve, said top plate aperture and said outflow check valve surrounded by said air outflow tube, said bottom plate including an aperture located within the confines of said accordion member and covered on its top surface by said air intake check valve, said compression spring captured within said accordion member and between the underside of said top plate and the top side of said bottom plate, said plenum portion terminating at its distal end in an air intake aperture, and said top plate limiting member located between said top plate and said bottom plate and being adjustable in length to selectively restrict the distance that said top plate travels toward said bottom plate.

### BRIEF DESCRIPTION OF THE DRAWINGS

FIG. 1 is a side section view of the invention.

FIG. 2 is a side view of the invention with the top plate in the high position.

FIG. 3 is a side view of the invention with the top plate in the medium position.

FIG. 4 is a side view of the invention with the top plate in the low position.

FIG. 5 is a top view of the invention.

FIG. 6 is a perspective view of the invention showing one handed operation.

FIG. 7 is a side view of the invention showing an alternate top plate travel restrictor.

FIG. 8 is a top view of the invention with a portion of the top plate cut away to reveal the cord retaining hook.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

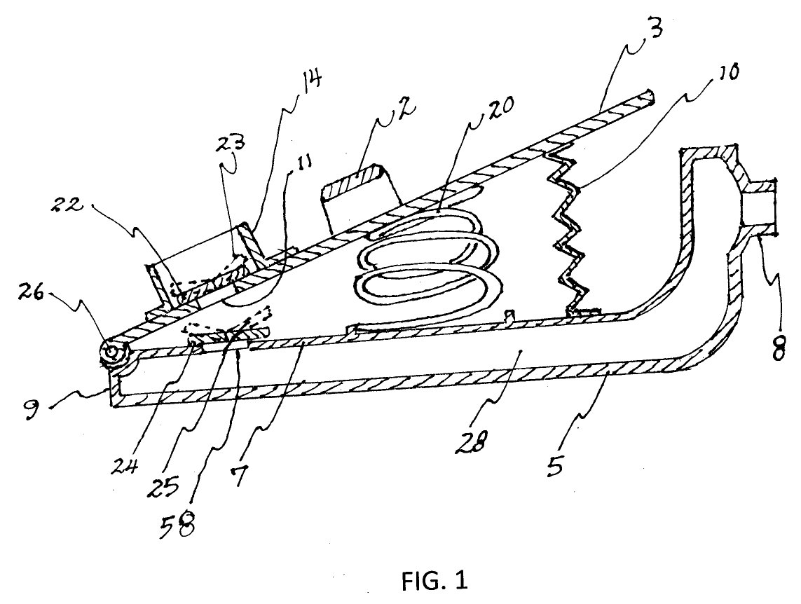

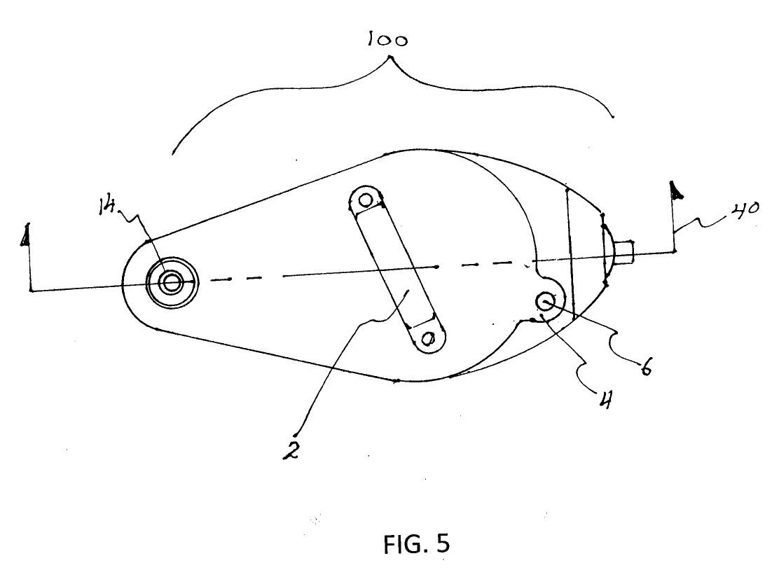

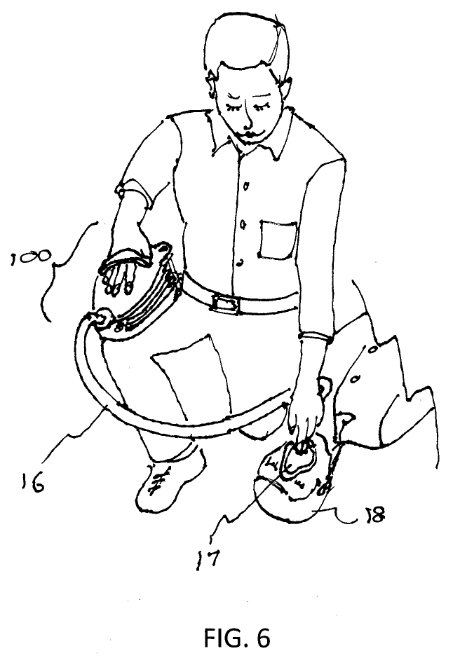

Referring now to FIG. 1 we see a section view of the invention 100 as defined by the section line 40 shown in FIG. 5. A rigid top plate 3 is hingedly attached by hinge pin 26 to a rigid bottom plate 7. An accordion member 10 is attached in an air tight manner to the under surface of top plate 10 and to the upper surface of bottom plate 7. The accordion member 10 is made of non porous material such as flexible urethane plastic. Another embodiment for the accordion member 10 includes flexible urethane coated nylon fabric such as the type found on a standard foot operated air pump. This alternate embodiment uses the flexible nature of the nylon fabric to compress down as the top panel 3 is pushed down. Bottom sub plate 5 and side surround 9 join with bottom plate 7 to form an air plenum 28. Bottom plate 7 and bottom sub plate 5 are parallel to each other in the present embodiment. When the top plate 3 is pushed down by the hand of a user such as a paramedic, air is forced out of top plate aperture 11 and through rubber check valve 22 causing the check valve to be pushed open as indicated by dotted line 23. Air then continues out of outflow tube 14 which is fixedly attached to top plate 3. The air then continues to the face mask 17 as shown in FIG. 6. The face mask 17 can also be directly attached to outflow tube 14. When the user releases downward force on the top plate 3, compression spring member 20 helps return the top plate 3 to its original position. Air is drawn into the plenum aperture 8 and up though aperture 58 and rubber check valve 24, shown in its open position by dotted lines 25, into the accordion 10 chamber so that the device 100 is ready for the next air cycle because the top plate 10, and bottom plate 7 are made of rigid material the volume of air delivered to the user with each push cycle is consistent and repeatable. Hand strap 2 also helps raise the top plate 3 and also allows the user to operate the device 100 with one hand while holding the face mask 17, with the other hand, onto the patient's nose and mouth as shown in FIG. 6.

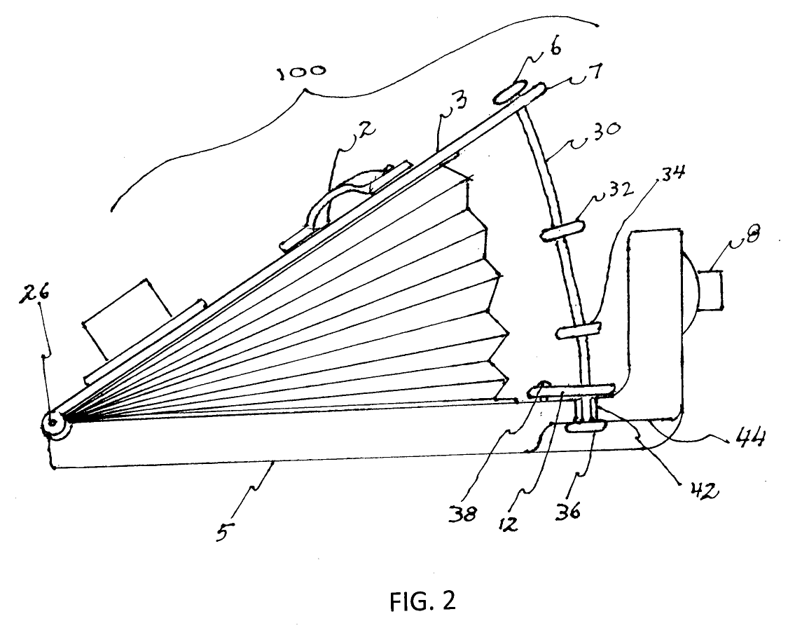

FIG. 2 shows a side view of the invention 100 with the top plate 3 in the raised position. Flexible cord 30 includes a plurality of spaced protuberances 6, 32, 34, 36 that help restrain the top plate's 3 movement. First protuberance 6 is located just above the top plate 3. The cord 30 passes through an aperture in the top plate 3 as shown by dotted line 7. In the present view the last protuberance 36 is retained in a slot 42 located on the side of the bottom plenum assembly. A recess 44 in the underside of the plenum assembly allows the protuberance 36 to be retained while still allowing the underside of the sub base plate 5 to be in contact with a ground plane. The current FIG. 2 shows the top plate in its upper most position. In this position the amount of air, about one thousand ml per stroke, within bellows portion 10 is appropriate for filling the lungs of an average adult, when the user repeatedly compresses top plate 3 to its fullest extent.

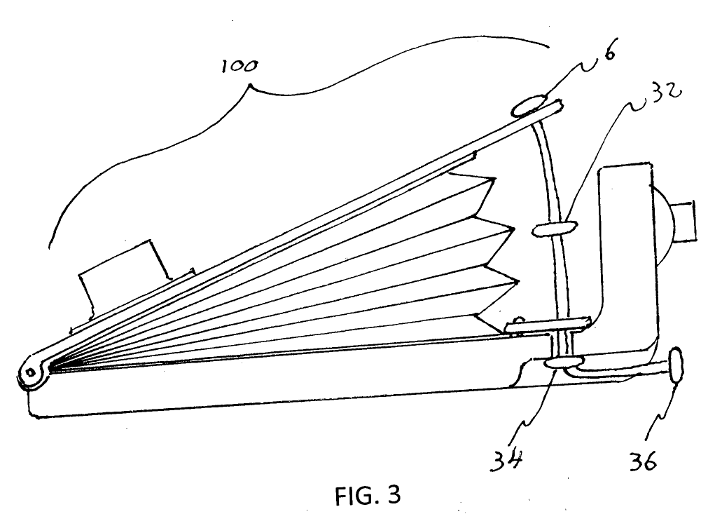

FIG. 3 shows the present invention 100 where the accordion member 10 and top plate 3 is set to an intermediate position because protuberance 34 is restricting the motion of top plate 3. The amount of air within the bellows 10, about four hundred and fifty ml per stroke, is now appropriate for a child so that each time the top plate is pushed, the proper amount of air is introduced into the child patient's lungs for optimal resuscitation activity.

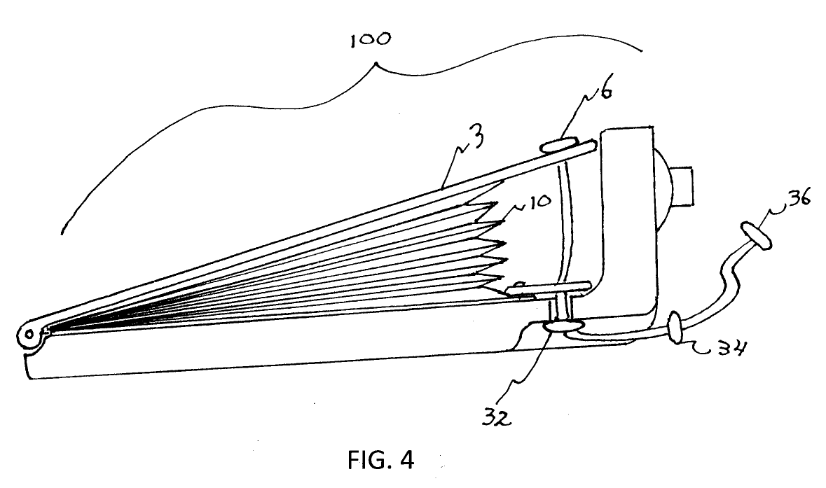

FIG. 4 shows the present invention 100 where the accordion member 10 and top plate 3 are set to a low position because protuberance 32 is restricting the motion of top plate 3. The amount of air within the bellows 10, about one hundred and fifty ml per stroke, is now appropriate for an infant so that each time the top plate is pushed the proper amount of air is introduced into the infant patient's lungs for optimal resuscitation activity. This lowest setting is also ideal for maintaining bellows 10 in its compressed position for compact storage and shipping.

FIG. 5 shows a top view of the invention 100 where hand strap 2 can be clearly seen, as well as tab 4 which extends from rigid top plate 3 and top restraining protuberance 6.

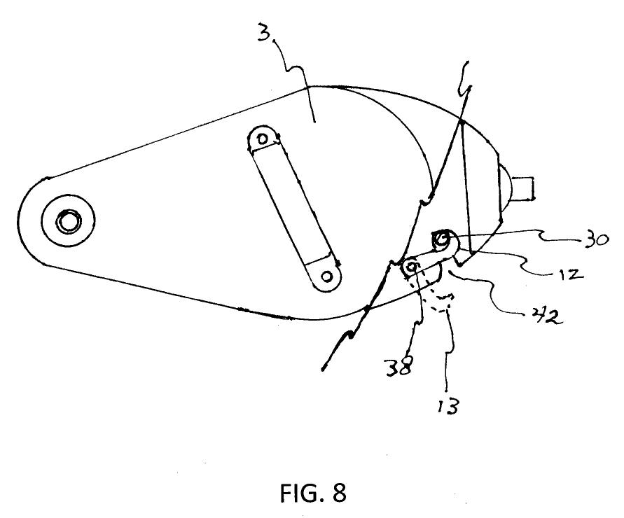

FIG. 8 shows a top view of the invention 100 with part of the top plate 3 removed to reveal cord 30 being retained by pivotable 38 hook 12. To change the restrained height of top plate 3, the user swings hook 12 out as shown by dotted line 13 and withdraws the cord 30 from slot 42 and resets the cord 30 to the desired height.

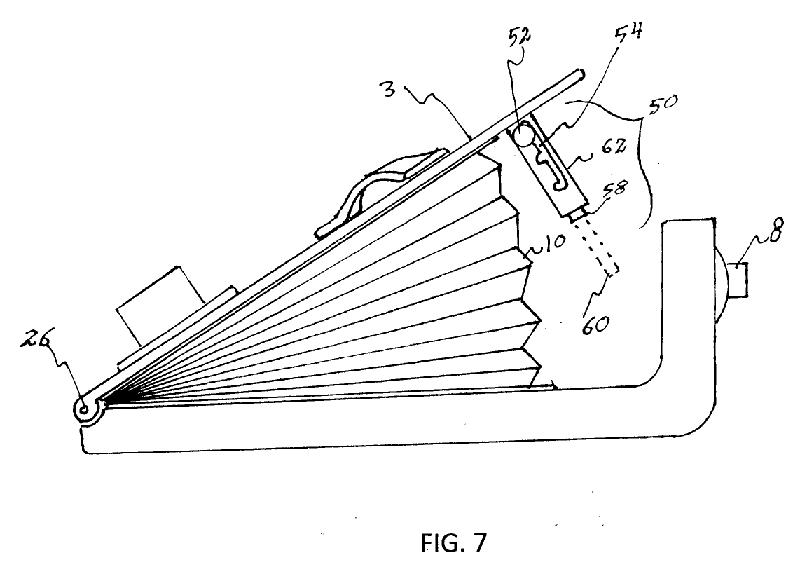

An alternate embodiment of a restrictor member for top plate 3 can be shown in FIG. 7 where a tube 62 retains a post 58 that can be adjusted in height as shown by dotted line 60. Thumb screw 52 holds the post 58 to the desired height. Slot 54 in tube 62 allows the threaded post of the thumb screw 52 to be slid down in a controlled way to the desired notch. Each notch represents a different degree of travel for accordion member 10. The top notch represents the adult setting. The middle notch represents the child setting. The lowest notch represents the infant setting. The post 58 takes the place of cord 30 in restricting the travel of top plate 3.

Referring back to FIG. 6, we see a perspective view of a patient 18 in the process of being resuscitated by the present invention 100. The invention 100 is a portable device that supplies air to the patient via flexible tube 16 and mouth/nose mask 17. The device 100 employs a bellows 10 (see FIG. 3) and a standard check valve pumping system to cause air to be delivered to a patient. The device 100 essentially replaces previous airway management resuscitation tools for bringing a non breathing patient back to life.

The above described and illustrated invention allows a user such as a paramedic to be ready to resuscitate an individual no matter what their lung capacity because the invention can be quickly set so that the air delivered to the patient is ideal and consistent for the lung size of the individual.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. resuscitator comprising:
a rigid top plate;
a rigid bottom plate;
a rigid bottom sub plate separated from and approximately parallel to said bottom plate;
a continuous rigid side surround panel that joins the perimeter edges of said bottom plate and said bottom sub plate forming an air flow plenum assembly;
said top plate hingedly joined at its forward end to said bottom plate;
a flexible non porous accordion member;
an air intake check valve;
an air outflow check valve;
an air out flow tube fixedly attached to the top surface of said top plate;
a compression spring;
a top plate travel limiting member;
the underside of said top plate joined to the upper side of said bottom plate by said accordion member;
said top plate including an aperture covered on its top surface by said air outflow check valve;
said top plate aperture and said outflow check valve surrounded by said air outflow tube;
said bottom plate including an aperture located within the confines of said accordion member and covered on its top surface by said air intake check valve;
said compression spring captured within said accordion member and between the underside of said top plate and the top side of said bottom plate;
said plenum portion terminating at its distal end in an air intake aperture; and
said top plate limiting member located between said top plate and said bottom plate and being adjustable in length to selectively restrict the distance that said top plate travels toward said bottom plate.

2. Resuscitator as claimed in claim 1 wherein said top plate travel limiting member is comprised of a length of cord that includes a plurality of spaced protuberances acting as restraining members;
one end of said cord fixedly attached to the distal end of said top plate;
the other end of said cord removably attached within a retaining slot located on the side of said bottom plenum assembly so that one said retaining member protuberance can be selected to reside at the underside of said slot causing the travel of said top plate to be limited to the distance defined by said retained cord.

3. Resuscitator as claimed in claim 1 further comprising a hinged hook shaped member that is rotatably pinned to the top surface of said bottom panel and removably retains said cord within said slot.

4. Resuscitator as claimed in claim 1 wherein said the compression of said accordion member causes a predetermined and repeatable volume of air to be discharged from said outflow tube from each complete stroke of said accordion compression.

5. Resuscitator as claimed in claim 1 wherein each said cord restraining protuberance corresponds to a human lung type;
said first protuberance corresponding to an infant lung type;
said second protuberance corresponding to a child lung type;
said third protuberance corresponding to an adult lung type.

6. Resuscitator as claimed in claim 1 wherein one said restraining protuberance holds said top plate and said accordion in a compressed state for purposes of compact storage.
